(19) United States
(12) Patent Application Publication
(10) Pub. No.: US 2017/0296587 A1
(43) Pub. Date: Oct. 19, 2017

(54) STEM CELL THERAPY OF NEUROLOGICAL MANIFESTATIONS OF A VIRAL INFECTION

(71) Applicant: CREATIVE MEDICAL HEALTH, INC, Phoenix, AZ (US)

(72) Inventors: Thomas Ichim, San Diego, CA (US); Amit Patel, Salt Lake City, UT (US)

(21) Appl. No.: 15/490,778

(22) Filed: Apr. 18, 2017

Related U.S. Application Data
(60) Provisional application No. 62/324,849, filed on Apr. 19, 2016.

(51) Int. Cl. A61K 35/28

(52) U.S. Cl. CPC A61K 35/28

(57) ABSTRACT

Disclosed are compositions of matter, protocols, and methods of treatment of neurological manifestations using stem cells, stem cell stimulators, and combination treatments. In one embodiment, a patient suffering neurological manifestations of a viral infection is administered a therapeutically active dose(s) of mesenchymal stem cells at a frequency and concentration sufficient to induce amelioration, remission or cure of neurological manifestations.

### CROSS-REFERENCE TO RELATED APPLICATIONS

The present invention claims priority to U.S. Provisional Application No. 62/324,849, filed Apr. 19, 2016, which is hereby incorporated in its entirety including all tables, figures, and claims.

### FIELD OF THE INVENTION

The invention pertains to the field of neurological abnormalities, more specifically, the invention pertains to the field of treatment of neurological abnormalities associated with infection of Zika Virus by administration of cellular therapies.

### BACKGROUND OF THE INVENTION

Zika virus is an arbovirus (arthropod borne virus). It is a member of the Flaviviridae family, genus Flavivirus, which includes dengue, yellow fever, and West Nile viruses. It was first identified in the Zika forest near Kampala, Uganda in rhesus macaques in 1947. Zika virus is a single stranded RNA virus with two major lineages: Asian and AfricanFew complete Zika virus genome sequences are available, and to date only two are from the current South American epidemic. Phylogenetic analysis of a Suriname Zika virus indicates that it belongs to the Asian genotype. It is most closely related to the strain that was circulating in French Polynesia in 2013, sharing more than 99.7% and 99.9% of nucleotide and amino acid identity, respectively.

Until recently, Zika virus was less of a research priority than other flaviviruses, as it was not thought to be of public health importance. Limited literature exists on the pathogenesis of the Zika virus to help understand the clinical disease spectrum and to target treatments to minimise or prevent tissue damage. Zika virus replicates readily in skin immune cells, and a large number of receptors are able to mediate entry of the virus into cells. Studies on the capability of the virus to replicate in neuronal cells are warranted to further investigate the link with neurological disorders.

Between the first isolation of Zika virus in monkeys in 1947 until 2007, reports of human cases were rare and sporadic. In 2007 an outbreak caused by a strain of Asian lineage occurred on the island of Yap, an island state of the Federated States of Micronesia. Estimated cases affected in this and subsequent outbreaks to date are probably imprecise, given the incomplete laboratory confirmation and the similarities in clinical presentation of Zika virus with other arbovirus infections present throughout the tropics. In Yap, 49 confirmed and 59 probable cases (defined according to strict serological criteria or RNA detection by reverse transcription-polymerase chain reaction) were identified over a four month period.

Because most people with Zika virus infection may not present for medical attention, estimating the total number of infected cases from the numbers of clinically suspected and laboratory confirmed cases is problematic. Estimates are influenced by the criteria used for definition of a suspected case and by assumptions made about the proportion of subclinical infections. Brazilian authorities estimate that around 1.5 million cases of Zika virus infection have occurred since the outbreak began. Unfortunately, to date, no treatment exists for the neurological manifestations and sequel of Zika Virus infection.

### DESCRIPTION OF THE INVENTION

The current invention discloses compositions of matters, protocols, and cells useful for the treatment of neurological manifestations of Zika Virus. In one embodiment the invention provides means of utilizing mesenchymal stem cells for treatment of neurological manifestations of Zika Virus. In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′10⁶ per 175 cm². Said bone marrow MSC may be administered intravenously, or in a preferred embodiment, intrathecally in a patient suffering from Zika Virus neurological manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In some embodiments of the invention MSC are transferred to possess enhanced neuromodulatory and neuroprotective properties. Said transfection may be accomplished by use of lentiviral vectors, said means to perform lentiviral mediated transfection are well-known in the art and discussed in the following references [1-7]. Some specific examples of lentiviral based transfection of genes into MSC include transfection of SDF-1 to promote stem cell homing, particularly hematopoietic stem cells [8], FGF-18 to promote osteogenic differentiation [9], GDNF to treat Parkinson's in an animal model [10], HGF to accelerate remyelination in a brain injury model [11], akt to protect against pathological cardiac remodeling and cardiomyocyte death [12], TRAIL to induce apoptosis of tumor cells [13-16], PGE-1 synthase for cardioprotection [17], NUR77 to enhance migration [18], BDNF to reduce ocular nerve damage in response to hypertension [19], HIF-1 alpha to stimulate osteogenesis [20], dominant negative CCL2 to reduce lung fibrosis [21], interferon beta to reduce tumor progression [22], HLA-G to enhance immune suppressive activity [23], hTERT to induce differentiation along the hepatocyte lineage [24], cytosine deaminase [25], OCT-4 to reduce senescence [26, 27], BAMBI to reduce TGF expression and protumor effects [28], HO-1 for cardioprotection [29], LIGHT to induce antitumor activity [30], miR-126 to enhance angiogenesis [31, 32], bcl-2 to induce generation of nucleus pulposus cells [33], telomerase and myocardin to induce cardiogenesis [34], CXCR4 to accelerate hematopoietic recovery [35] and reduce renal allograft rejection [36], wnt11to promote chondrogenesis [37], Islet-1 to promote pancreatic differentiation [38], IL-27 to reduce autoimmune disease [39], ACE-2 to reduce sepsis [40], CXCR4 to reduce liver failure [41], and lung injury [42], and the HGF antagonist NK4 to reduce cancer [43].

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the invention MSC are transfected with anti-apoptotic proteins to enhance in vivo longevity. The present invention includes a method of using MSC that have been cultured under conditions to express increased amounts of at least one anti-apoptotic protein as a therapy to inhibit or prevent apoptosis. In one embodiment, the MSC which are used as a therapy to inhibit or prevent apoptosis have been contacted with an apoptotic cell. The invention is based on the discovery that MSC that have been contacted with an apoptotic cell express high levels of anti-apoptotic molecules. In some instances, the MSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MSC have been previously described which can be applied to the current invention, said antiapoptotic genes that can be utilized for practice of the invention, in a nonlimiting way, include GATA-4 [44], FGF-2 [45], bcl-2 [33, 46], and HO-1 [47]. Based upon the disclosure provided herein, MSC can be obtained from any source. The MSC may be autologous with respect to the recipient (obtained from the same host) or allogeneic with respect to the recipient. In addition, the MSC may be xenogeneic to the recipient (obtained from an animal of a different species). In one embodiment of the invention MSC are pretreated with agents to induce expression of antiapoptotic genes, one example is pretreatment with exendin-4 as previously described [48]. In a further non-limiting embodiment, MSC used in the present invention can be isolated, from the bone marrow of any species of mammal, including but not limited to, human, mouse, rat, ape, gibbon, bovine. In a non-limiting embodiment, the MSC are isolated from a human, a mouse, or a rat. In another non-limiting embodiment, the MSC are isolated from a human.

Based upon the present disclosure, MSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MSC are cultured in a manner that provides MSC to express increased amounts of at least one anti-apoptotic protein. Culture conditions for growth of clinical grade MSC have been described in the literature and are incorporated by reference [49-82].

Any medium capable of supporting MSC in vitro may be used to culture the MSC. Media formulations that can support the growth of MSC include, but are not limited to, Dulbecco's Modified Eagle's Medium (DMEM), alpha modified Minimal Essential Medium (.alpha.MEM), and Roswell Park Memorial Institute Media 1640 (RPMI Media 1640) and the like. Said media and conditions for culture of MSC-and by virtue of the invention MSC are known in the art. Typically, up to 20% fetal bovine serum (FBS) or 1-20% horse serum is added to the above medium in order to support the growth of MSC. A defined medium, however, also can be used if the growth factors, cytokines, and hormones necessary for culturing MSC are provided at appropriate concentrations in the medium. Media useful in the methods of the invention may contain one or more compounds of interest, including, but not limited to, antibiotics, mitogenic or differentiation compounds useful for the culturing of MSC. The cells may be grown at temperatures between 27.degree. C. to 40.degree. C., preferably 31.degree. C. to 37.degree. C., and more preferably in a humidified incubator. The carbon dioxide content may be maintained between 2% to 10% and the oxygen content may be maintained between 1% and 22%. The invention, however, should in no way be construed to be limited to any one method of isolating and culturing MSC. Rather, any method of isolating and culturing MSC should be construed to be included in the present invention.

Antibiotics which can be added into the medium include, but are not limited to, penicillin and streptomycin. The concentration of penicillin in the culture medium, in a non-limiting embodiment, is about 10 to about 200 units per ml. The concentration of streptomycin in the culture medium is, in a non-limiting embodiment, about 10 to about 200 .mu.g/ml.

MSC which express increased amounts of at least one anti-apoptotic protein may be administered to an animal in an amount effective to provide a therapeutic effect. The animal may be a mammal, including but not limited to, human and non-human primates.

The MSC can be suspended in an appropriate diluent. Suitable excipients for injection solutions are those that are biologically and physiologically compatible with the MSC and with the recipient, such as buffered saline solution or other suitable excipients. The composition for administration can be formulated, produced, and stored according to standard methods complying with proper sterility and stability. The MSC may have one or more genes modified or be treated such that the modification has the ability to cause the MSC to self-destruct or “commit suicide” because of such modification, or upon presentation of a second drug (eg., a prodrug) or signaling compound to initiate such destruction of the MSC.

In one embodiment of the invention, MSC are isolated from the subepithelial layer (SL) of the umbilical cord. Isolated cells from the SL can have a variety of characteristic markers that distinguish them from cell previously isolated from umbilical cord samples. It should be noted that these isolated cells are not derived from the Wharton's Jelly, but rather from the SL. Various cellular markers that are either present or absent can be utilized in the identification of these SL-derived cells, and as such, can be used to show the novelty of the isolated cells. For example, in one aspect, the isolated cell expresses at least three cell markers selected from CD29, CD73, CD90, CD146, CD166, SSEA4, CD9, CD44, CD146, or CD105, and the isolated cell does not express at least three cell markers selected from CD45, CD34, CD14, CD79, CD106, CD86, CD80, CD19, CD117, Stro-1, or HLA-DR. In another aspect, the isolated cell expresses at least five cell markers selected from CD29, CD73, CD90, CD146, CD166, SSEA4, CD9, CD44, CD146, or CD105. In another aspect, the isolated cell expresses at least eight cell markers selected from CD29, CD73, CD90, CD146, CD166, SSEA4, CD9, CD44, CD146, or CD105. In a yet another aspect, the isolated cell expresses at least CD29, CD73, CD90, CD166, SSEA4, CD9, CD44, CD146, and CD105. In another aspect, the isolated cell does not express at least five cell markers selected from CD45, CD34, CD14, CD79, CD106, CD86, CD80, CD19, CD117, Stro-1, or HLA-DR. In another aspect, the isolated cell does not express at least eight cell markers selected from CD45, CD34, CD14, CD79, CD106, CD86, CD80, CD19, CD117, Stro-1, or HLA-DR. In yet another aspect, the isolated cell does not express at least CD45, CD34, CD14, CD79, CD106, CD86, CD80, CD19, CD117, Stro-1, and HLA-DR. Additionally, in some aspects, the isolated cell can be positive for SOX2, OCT4, or both SOX2 and OCT4. In a further aspect, the isolated cell can produce exosomes expressing CD63, CD9, or both CD63 and CD9.

A variety of techniques can be utilized to extract the isolated cells of the present disclosure from the SL, and any such technique that allows such extraction without significant damage to the cells is considered to be within the present scope. In one aspect, for example, a method of culturing stem cells from the SL of a mammalian umbilical cord can include dissecting the subepithelial layer from the umbilical cord. In one aspect, for example, umbilical cord tissue can be collected and washed to remove blood, Wharton's Jelly, and any other material associated with the SL. For example, in one non-limiting aspect the cord tissue can be washed multiple times in a solution of Phosphate-Buffered Saline (PBS) such as Dulbecco's Phosphate-Buffered Saline (DPBS). In some aspects the PBS can include a platelet lysate (i.e. 10% PRP lysate of platelet lysate). Any remaining Wharton's Jelly or gelatinous portion of the umbilical cord can then be removed and discarded. The remaining umbilical cord tissue (the SL) can then be placed interior side down on a substrate such that an interior side of the SL is in contact with the substrate. An entire dissected umbilical cord with the Wharton's Jelly removed can be placed directly onto the substrate, or the dissected umbilical cord can be cut into smaller sections (e.g. 1-3 mm) and these sections can be placed directly onto the substrate.

A variety of substrates are contemplated upon which the SL can be placed. In one aspect, for example, the substrate can be a solid polymeric material. One example of a solid polymeric material can include a cell culture dish. The cell culture dish can be made of a cell culture treated plastic as is known in the art. In one specific aspect, the SL can be placed upon the substrate of the cell culture dish without any additional pretreatment to the cell culture treated plastic. In another aspect, the substrate can be a semi-solid cell culture substrate. Such a substrate can include, for example, a semi-solid culture medium including an agar or other gelatinous base material. Following placement of the SL on the substrate, the SL is cultured in a suitable medium. In some aspects it is preferable to utilized culture media that is free of animal and human components or contaminants. As one example, FIG. 2 shows the culturing of cells from the SL.

The culture can then be cultured under either normoxic or hypoxic culture conditions for a period of time sufficient to establish primary cell cultures. (e.g. 3-7 days in some cases). After primary cell cultures have been established, the SL tissue is removed and discarded. Cells or stem cells are further cultured and expanded in larger culture flasks in either a normoxic or hypoxic culture conditions. While a variety of suitable cell culture media are contemplated, in one non-limiting example the media can be Dulbecco's Modified Eagle Medium (DMEM) glucose (500-6000 mg/mL) without phenol red, 1.times. glutamine, 1.times.NEAA, and 0.1-20% PRP lysate or platelet lysate. Another example of suitable media can include a base medium of DMEM low glucose without phenol red, 1.times. glutamine, 1.times.NEAA, 1000 units of heparin and 20% PRP lysate or platelet lysate. In another example, cells can be cultured directly onto a semi-solid substrate of DMEM low glucose without phenol red, 1.times. glutamine, 1.times.NEAA, and 20% PRP lysate or platelet lysate. In a further example, culture media can include a low glucose medium (500-1000 mg/mL) containing 1.times. Glutamine, 1.times.NEAA, 1000 units of heparin. In some aspects, the glucose can be 1000-4000 mg/mL, and in other aspects the glucose can be high glucose at 4000-6000 mg/mL. These media can also include 0.1%-20% PRP lysate or platelet lysate. In yet a further example, the culture medium can be a semi-solid with the substitution of acid-citrate-dextrose ACD in place of heparin, and containing low glucose medium (500-1000 mg/mL), intermediate glucose medium (1000-4000 mg/mL) or high glucose medium (4000-6000 mg/mL), and further containing 1.times. Glutamine, 1.times.NEAA, and 0.1%-20% PRP lysate or platelet lysate. In some aspects, the cells can be derived, subcultured, and/or passaged using TrypLE. In another aspect, the cells can be derived, subcultured, and/or passaged without the use of TrypLE or any other enzyme.

In one aspect, SL cells can be cultured from a mammalian umbilical cord in a semi-solid PRP Lysate or platelet lysate substrate. Such cells can be cultured directly onto a plastic coated tissue culture flask as has been described elsewhere herein. After a sufficient time in either normoxic or hypoxic culture environments the media is changed and freshly made semi-solid PRP lysate or platelet lysate media is added to the culture flask. The flask is continued to be cultured in either a normoxic or hypoxic culture environment. The following day the media becomes a semi-solid PRP-lysate or platelet lysate matrix. The cells can be continued to be cultured in this matrix being until further use. In one specific aspect, ingredients for a semi solid culture can include growth factors for expanded cell culture of differentiation. Non-limiting examples can include FGF, VEGF, FNDC5, 5-azacytidine, TGF-Beta1, TGF Beta2, insulin, ITS, IGF, and the like, including combinations thereof.

In some cases, allogenic confirmation of SL cells, either differentiated or undifferentiated, can be highly beneficial, particularly for therapeutic uses of the cells. In such cases, mixed lymphocyte reactions can be performed on the cells to confirm the allogenic properties of the cells. In certain aspects, a cell derived as described herein does not cause a mixed lymphocyte response or T-cell proliferation.

In certain aspects, a cell derived as described herein can be recombinantly modified to express one or more genes and or proteins. In one technique, a gene or genes can be incorporated into an expression vector. Approaches to deliver a gene into the cell can include without limitation, viral vectors, including recombinant retroviruses, adenoviruses, adeno-associated virus, lentivirus, poxivirus, alphavirus, herpes simplex virus-1, recombinant bacterial, eukaryotic plasmids, and the like, including combinations thereof. Plasmid DNA may be delivered naked or with the help of exosomes, cationic liposomes or derivatized (antibody conjugated) polylysine conjugates, gramicidin S, artificial viral envelopes, other intracellular carriers, as well as direct injection of the genes. In some aspects, non-viral gene delivery methods can be used, such as for example, scaffold/matrix attached region (S/MAR)-based vector.

Furthermore, in some aspects, isolated SL cells can be used to produce an exosome population. These exosome populations can be utilized for a variety of research and therapeutic uses. In one aspect, for example, cells are cultured as described in either a normoxic or hypoxic culture environment and supernatants are collected at each media change. Exosomes can then be purified from the supernatants using an appropriate purification protocol. One not-limiting example of such a protocol is the ExoQuick isolation system by SYSTEMBIO. Purified exosomes can be utilized for further manipulation, targeting, and therapeutic use. The exosomes specific to the SL cells are positive for CD63 expression. The dosage of the MSC varies within wide limits and may be adjusted to the individual requirements in each particular case. The number of cells used depends on the age, weight, sex, and condition of the recipient, the number and/or frequency of administrations, the disease or disorder being treated, and the extent or severity thereof, and other variables known to those of skill in the art.

The amniotic fluid-derived stem cells described in this invention are capable of self-renewal in tissue culture, maintain euploidy for >1 year in culture, share markers with human ES cells, and are capable of differentiating into all three germ layers of the developing embryo, Endoderm, Mesoderm and Ectoderm. In a preferred embodiment the regenerative amniotic fluid cells are found in the amnion harvested during the second trimester of human pregnancies. It is known that amniotic fluid contains multiple morphologically-distinguishable cell types, the majority of the cells are prone to senescence and are lost from cultures. In one embodiment, fibronectin coated plates and culture conditions described in U.S. Pat. No. 7,569,385 are used to grow cells from amniotic fluid harvests from normal 16-18 week pregnancies. The cells of the invention are of fetal origin, and have a normal diploid karyotype. Growth of the amniotic fluid stem cells as described in the invention for use in neurological ischemic conditions results in cells that are multipotent, as several main cell types have been derived from them. As used herein, the term “multipotent” refers to the ability of amniotic fluid regenerative cells to differentiate into several main cell types. The MAFSC cells may also be propagated under specific conditions to become “pluripotent.” The term “pluripotent stem cells” describes stem cells that are capable of differentiating into any type of body cell, when cultured under conditions that give rise to the particular cell type. The Amniotic fluid regenerative cells are preferably isolated from humans. However, the Amniotic fluid regenerative cells may be isolated in a similar manner from other species. Examples of species that may be used to derive the Amniotic fluid regenerative cells include but are not limited to mammals, humans, primates, dogs, cats, goats, elephants, endangered species, cattle, horses, pigs, mice, rabbits, and the like.

The amniotic fluid-derived cells and MAFSC can be recognized by their specific cell surface proteins or by the presence of specific cellular proteins. Typically, specific cell types have specific cell surface proteins. These surface proteins can be used as “markers” to determine or confirm specific cell types. Typically, these surface markers can be visualized using antibody-based technology or other detection methods.

The surface markers of the isolated MAFSC cells derived from independently-harvested amniotic fluid samples were tested for a range of cell surface and other markers, using monoclonal antibodies and FACS analysis. These cells can be characterized by the following cell surface markers: SSEA3, SSEA4, Tra-1-60, Tra-1-81, Tra-2-54. The MAFSC cells can be distinguished from mouse ES cells in that the MAFSC cells do not express the cell surface marker SSEA1. Additionally, MAFSC express the stem cell transcription factor Oct-4. The MAFSC cells can be recognized by the presence of at least one, or at least two, or at least three, or at least four, or at least five, or at least six, or all of the following cellular markers SSEA3, SSEA4, Tra-1-60, Tra-1-81, Tra-2-54 and Oct-4.

The MAFSC cultures express very little or no SSEA-1 marker. In addition to the embryo stem cell markers SSEA3, SSEA4, Tra1-60, Tra1-81, Tra2-54, Oct-4 the amniotic fluid regenerative cells also expressed high levels of the cell surface antigens that are normally found on human mesenchymal stem cells, but not normally on human embryo stem cells. This set of markers includes CD13 (99.6%) aminopeptidase N, CD44 (99.7%) hyaluronic acid-binding receptor, CD49b (99.8%) collagen/laminin-binding integrin alpha2, and CD105 (97%) endoglin. The presence of both the embryonic stem cell markers and the hMSC markers on the MAFSC cell cultures indicates that amniotic fluid-derived MAFSC cells, grown and propagated as described here, represent a novel class of human stem cells that combined the characteristics of hES cells and of hMSC cells.

In some embodiments of the invention, at least about 90%, 94%, 97%, 99%, or 100% of the cells in the culture express CD13. In additional embodiments, at least about 90%, 94%, 97%, 99%, or 100% of the cells in the culture express CD44. In some embodiments of the invention, a range from at least about 90%, 94%, 97%, 99%, 99.5%, or 100% of the cells in the culture express CD49b. In further embodiments of the invention, a range from at least about 90%, 94%, 97%, 99%, 99.5%, or 100% of the cells in the culture express CD105.

In one particular embodiment of the invention, the amniotic fluid regenerative cells are human stem cells that can be propagated for an indefinite period of time in continuous culture in an undifferentiated state. The term “undifferentiated” refers to cells that have not become specialized cell types. A “nutrient medium” is a medium for culturing cells containing nutrients that promote proliferation. The nutrient medium may contain any of the following in an appropriate combination: isotonic saline, buffer, amino acids, antibiotics, serum or serum replacement, and exogenously added factors.

In one embodiment, mesenchymal stem cells used are amniotic fluid stem cells. Said Amniotic fluid stem cells, or “regenerative cells” may be grown in an undifferentiated state for as long as desired (and optionally stored as described above), and can then be cultured under certain conditions to allow progression to a differentiated state. By “differentiation” is meant the process whereby an unspecialized cell acquires the features of a specialized cell such as a heart, liver, muscle, pancreas or other organ or tissue cell. The Amniotic fluid regenerative cells, when cultured under certain conditions, have the ability to differentiate in a regulated manner into three or more subphenotypes. Once sufficient cellular mass is achieved, cells can be differentiated into endodermal, mesodermal and ectodermal derived tissues in vitro and in vivo. This planned, specialized differentiation from undifferentiated cells towards a specific cell type or tissue type is termed “directed differentiation.” Exemplary cell types that may be prepared from Amniotic fluid regenerative cells using directed differentiation include but are not limited to fat cells, cardiac muscle cells, epithelial cells, liver cells, brain cells, blood cells, neurons, glial cells, pancreatic cells, and the like.

General methods relating to stem cell differentiation techniques that may be useful for differentiating the Amniotic fluid regenerative cells of this invention can be found in general texts such as: Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998); and in Stem cell biology (L. M. Reid, Curr. Opinion Cell Biol. 2:121, 1990), each of which is incorporated by reference herein in its entirety.

In one embodiment of the invention, the MAFSC are allowed to adhere for 72 h followed by media changes every 3-4 days. Adherent cells are removed with 0.05% trypsin-EDTA and replated at a density of 1′10⁶ per 175 cm². Said MAFSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In some embodiments of the invention MAFSC are transfected to possess enhanced neuromodulatory and neuroprotective properties. Said transfection may be accomplished by use of lentiviral vectors, said means to perform lentiviral mediated transfection are well-known in the art and discussed in the following references [10-16]. Some specific examples of lentiviral based transfection of genes into MSC, which by include transfection of SDF-1 to promote stem cell homing, particularly hematopoietic stem cells [17],GDNF to treat Parkinson's in an animal model [18], HGF to accelerate remyelination in a brain injury model [19], akt to protect against pathological cardiac remodeling and cardiomyocyte death [20], TRAIL to induce apoptosis of tumor cells [21-24], PGE-1 synthase for cardioprotection [25], NUR77 to enhance migration [26], BDNF to reduce ocular nerve damage in response to hypertension [27], HIF-1 alpha to stimulate osteogenesis [28], dominant negative CCL2 to reduce lung fibrosis [29], interferon beta to reduce tumor progression [30], HLA-G to enhance immune suppressive activity [31], hTERT to induce differentiation along the hepatocyte lineage [32], cytosine deaminase [33], OCT-4 to reduce senescence [34, 35], BAMBI to reduce TGF expression and protumor effects [36], HO-1 for radioprotection [37], LIGHT to induce antitumor activity [38], miR-126 to enhance angiogenesis [39, 40], bcl-2 to induce generation of nucleus pulposus cells [41], telomerase to induce neurogenesis [42], CXCR4 to accelerate hematopoietic recovery [43] and reduce unwanted immunity [44], wnt11 to promote regenerative cytokine production [45], and the HGF antagonist NK4 to reduce cancer [46].

Cell cultures are tested for sterility weekly, endotoxin by limulus amebocyte lysate test, and mycoplasma by DNA-fluorochrome stain.

In order to determine the quality of MAFSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199 +1% human serum albumin (HSA; American Red Cross, Washington D.C., USA). MSCs harvested from each 10-flask set were stored for up to 4 h at 4° C. and combined at the end of the harvest. A total of 2-10′10⁶ MSC/kg were resuspended in M199+1% HSA and centrifuged at 460 g for 10 min at 20° C. Cell pellets were resuspended in fresh M199 +1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the invention MAFSC are transfected with anti-apoptotic proteins to enhance in vivo longevity. The present invention includes a method of using MAFSC that have been cultured under conditions to express increased amounts of at least one anti-apoptotic protein as a therapy to inhibit or prevent apoptosis. In one embodiment, the MAFSC which are used as a therapy to inhibit or prevent apoptosis have been contacted with an apoptotic cell. The invention is based on the discovery that MAFSC that have been contacted with an apoptotic cell express high levels of anti-apoptotic molecules. In some instances, the MAFSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MAFSC have been previously described which can be applied to the current invention, said antiapoptotic genes that can be utilized for practice of the invention, in a nonlimiting way, include GATA-4 [47], FGF-2 [48], bcl-2 [41, 49], and HO-1 [50]. Based upon the disclosure provided herein, MAFSC can be obtained from any source. The MAFSC may be autologous with respect to the recipient (obtained from the same host) or allogeneic with respect to the recipient. In addition, the MAFSC may be xenogeneic to the recipient (obtained from an animal of a different species). In one embodiment of the invention MAFSC are pretreated with agents to induce expression of antiapoptotic genes, one example is pretreatment with exendin-4 as previously described [51]. In a further non-limiting embodiment, MAFSC used in the present invention can be isolated, from the bone marrow of any species of mammal, including but not limited to, human, mouse, rat, ape, gibbon, bovine. In a non-limiting embodiment, the MAFSC are isolated from a human, a mouse, or a rat. In another non-limiting embodiment, the MAFSC are isolated from a human.

Based upon the present disclosure, MAFSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MAFSC are cultured in a manner that provides MAFSC to express increased amounts of at least one anti-apoptotic protein. Culture conditions for growth of clinical grade MSC, which under the scope of the invention MAFSC fall under, have been described in the literature and are incorporated by reference [52-85].

In one embodiment of the invention MAFSC are administered subcutaneously or intramuscularly in order to endow radiation production by secretion of soluble factors. In another embodiment soluble factors are isolated from said MAFSC and administered to patients in need of therapy. In another embodiment MAFSC are stimulated in vitro to produce soluble factors by treatment with hypoxia.

This invention calls for the administration of stem cells or neuronal cells to individuals experiencing the effects of CNS neuron loss. The cells can be administered a variety of ways: The cells of the present invention are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including, but not limited to, improved cognition, survival rate or more rapid recovery, or improvement or elimination of symptoms and other indicators as are selected as appropriate measures by those skilled in the art. In the method of the present invention, the cells of the present invention can be administered in various ways as would be appropriate to implant in the central nervous system. The amniotic fluid stem cells can be administered intracisternally, parenchymally, intravenously, intraventricularly or by other convenient means.

Various aspects of the invention relating to the above are enumerated in the following paragraphs:

Aspect 1. A method of treating neurological manifestations of a viral infection comprising the steps of: a) obtaining a patient suffering from a viral infection neurological manifestation; b) administering a therapeutic dose of stem cells; c) assessing effect of said stem cell administration; and d) performing additional administrations of said stem cells based on neurological response achieved.

Aspect 2. The method of claim 1, wherein said stem cells are selected from a group comprising of: a) adipose derived stem cells; b) embryonic stem cells; c) inducible pluripotent stem cells; d) hematopoietic stem cells; and e) mesenchymal stem cells.

Aspect 3. The method of claim 2, wherein said mesenchymal stem cell is derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

Aspect 4. The method of claim 3, wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Aspect 5. The method of claim 4, wherein said mesenchymal stem cells do not express substantial levels of HLA-DR, CD117, and CD45.

Aspect 6. The method of claim 3, wherein said mesenchymal stem cells are generated from a pluripotent stem cell.

Aspect 7. The method of claim 6, wherein said pluripotent stem cell is selected from a group comprising of: a) an embryonic stem cell; b) an inducible pluripotent stem cell; c) a parthenogenic stem cell; and d) a somatic cell nuclear transfer derived stem cell.

Aspect 8. The method of claim 7, wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Aspect 9. The method of claim 7, wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.

Aspect 10. The method of claim 7, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Aspect 11. The method of claim 7, wherein said somatic cell nuclear transfer derived stem cells possess a phenotype negative for SSEA-1 and positive for SSEA-3, SSEA-4, TRA-1-60, TRA-1-81, and alkaline phosphatase.

Aspect 12. The method of claim 6, wherein said mesenchymal stem cells are differentiated from a pluripotent stem cell source through culture in the presence of an inhibitor of the SMAD-2/3 pathway.

Aspect 13. The method of claim 12, wherein said mesenchymal stem cells are differentiated from a pluripotent stem cell source through culture in the presence of an inhibitor nucleic acid targeting the SMAD-2/3 pathway.

Aspect 14. The method of claim 13, wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme.

Aspect 15. The method of claim 13, wherein said inhibitor of the SMAD-2/3 pathway is a small molecule inhibitor.

Aspect 16. The method of claim 15, wherein said small molecule inhibitor is SB-431542.

Aspect 17. The method of claim 6, wherein a selection process is used to enrich for mesenchymal stem cells differentiated from said pluripotent stem cell population.

Aspect 18. The method of claim 17, wherein said enrichment method comprises of positively selecting for cells expressing a marker associated with mesenchymal stem cells.

Aspect 19. The method of claim 18, wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Aspect 20. The method of claim 1, wherein said immune modulatory cells are autologous, allogeneic or xenogeneic to the recipient.

Aspect 21. The method of claim 2 wherein one or more cells are co-administered to said recipient based on specific need for immune modulation in said recipient.

Aspect 22. The method of claim 2, wherein an antigen is administered in combination with immune modulatory cells.

Aspect 23. The method of claim 1, wherein said stem cells is a cell obtained from a subepithelial layer of a mammalian umbilical cord tissue capable of self-renewal and culture expansion; wherein the isolated cell expresses at least three cell markers selected from the group consisting of CD29, CD73, CD90, CD166, SSEA4, CD9, CD44, CD146, or CD105; and wherein the isolated cell does not express at least three cell markers selected from the group consisting of CD45, CD34, CD14, CD79, CD106, CD86, CD80, CD19, CD117, Stro-1, or HLA-DR.

Aspect 24. The method of claim 23, wherein said isolated cell expresses CD29, CD73, CD90, CD166, SSEA4, CD9, CD44, CD146, and CD105.

Aspect 25. The method of claim 23, wherein said isolated cell does not express CD45, CD34, CD14, CD79, CD106, CD86, CD80, CD19, CD117, Stro-1, and HLA-DR.

Aspect 26. The method of claim 23, wherein said isolated cell expresses one or more markers selected from a group comprising of: a) SOX2; b) OCT-4; c) NANOG; and d) KLF-4.

Aspect 27. The method of claim 1, wherein said stem cells are treated with neurotrophic factors prior to administration.

Aspect 28. The method of claim 27, wherein said neurotrophic factors are selected from a group comprising of: a) NGF; b) HGF-1; c) BDNF; and d) FGF-5.

Aspect 29. The method of claim 1, wherein exosomes are derived from said stem cells and used together, or in substitution of said stem cells.

Aspect 30. A method of treating neurological manifestations of a viral infection through reducing microglial production of quinolinic acid through administration of mesenchymal stem cells.

Aspect 31. The method of claim 1, wherein said mesenchymal stem cells are placentally derived and express CD34 upon isolation.

### REFERENCES

1. Zhang, X. Y., et al., Lentiviral vectors for sustained transgene expression in human bone marrow-derived stromal cells. Mol Ther, 2002. 5(5 Pt 1): p. 555-65.

2. Kyriakou, C. A., et al., Human mesenchymal stem cells (hMSCs) expressing truncated soluble vascular endothelial growth factor receptor (tsFlk-1) following lentiviral-mediated gene transfer inhibit growth of Burkitt's lymphoma in a murine model. J Gene Med, 2006. 8(3): p. 253-64.

3. Worsham, D. N., et al., In vivo gene transfer into adult stem cells in unconditioned mice by in situ delivery of a lentiviral vector. Mol Ther, 2006. 14(4): p. 514-24.

4. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.

5. Kallifatidis, G., et al., Improved lentiviral transduction of human mesenchymal stem cells for therapeutic intervention in pancreatic cancer. Cancer Gene Ther, 2008. 15(4): p. 231-40.

6. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.

7. McGinley, L., et al., Lentiviral vector mediated modification of mesenchymal stem cells & enhanced survival in an in vitro model of ischaemia. Stem Cell Res Ther, 2011. 2(2): p. 12.

8. Liang, X., et al., Human bone marrow mesenchymal stem cells expressing SDF-1 promote hematopoietic stem cell function of human mobilised peripheral blood CD34+ cells in vivo and in vitro. Int J Radiat Biol, 2010. 86(3): p. 230-7.

9. Hamidouche, Z., et al., Autocrine fibroblast growth factor 18 mediates dexamethasone-induced osteogenic differentiation of murine mesenchymal stem cells. J Cell Physiol, 2010. 224(2): p. 509-15.

10. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.

11. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.

12. Yu, Y. S., et al., AKT-modified autologous intracoronary mesenchymal stem cells prevent remodeling and repair in swine infarcted myocardium. Chin Med J (Engl), 2010. 123(13): p. 1702-8.

13. Mueller, L. P., et al., TRAIL-transduced multipotent mesenchymal stromal cells (TRAIL-MSC) overcome TRAIL resistance in selected CRC cell lines in vitro and in vivo. Cancer Gene Ther, 2011. 18(4): p. 229-39.

14. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.

15. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.

16. Sage, E. K., et al., Systemic but not topical TRAIL-expressing mesenchymal stem cells reduce tumour growth in malignant mesothelioma. Thorax, 2014. 69(7): p. 638-47.

17. Lian, W. S., et al., In vivo therapy of myocardial infarction with mesenchymal stem cells modified with prostaglandin I synthase gene improves cardiac performance in mice. Life Sci, 2011. 88(9-10): p. 455-64.

18. Maijenburg, M. W., et al., Nuclear receptors Nur77 and Nurr1 modulate mesenchymal stromal cell migration. Stem Cells Dev, 2012. 21(2): p. 228-38.

19. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.

20. Zou, D., et al., In vitro study of enhanced osteogenesis induced by HIF-1alpha-transduced bone marrow stem cells. Cell Prolif, 2011. 44(3): p. 234-43.

21. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.

22. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.

23. Yang, H. M., et al., Enhancement of the immunosuppressive effect of human adipose tissue-derived mesenchymal stromal cells through HLA-G1 expression. Cytotherapy, 2012. 14(1): p. 70-9.

24. Liang, X. J., et al., Differentiation of human umbilical cord mesenchymal stem cells into hepatocyte-like cells by hTERT gene transfection in vitro. Cell Biol Int, 2012. 36(2): p. 215-21.

25. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.

26. Jaganathan, B. G. and D. Bonnet, Human mesenchymal stromal cells senesce with exogenous OCT4. Cytotherapy, 2012. 14(9): p. 1054-63.

27. Han, S. H., et al., Effect of ectopic OCT4 expression on canine adipose tissue-derived mesenchymal stem cell proliferation. Cell Biol Int, 2014. 38(10): p. 1163-73.

28. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.

29. Kearns-Jonker, M., et al., Genetically Engineered Mesenchymal Stem Cells Influence Gene Expression in Donor Cardiomyocytes and the Recipient Heart. J Stem Cell Res Ther, 2012. S1.

30. Ma, G. L., et al., [Study of inhibiting and killing effects of transgenic LIGHT human umbilical cord blood mesenchymal stem cells on stomach cancer]. Zhonghua Wei Chang Wai Ke Za Zhi, 2012. 15(11): p. 1178-81.

31. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.

32. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.

33. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.

34. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.

35. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.

36. Cao, Z., et al., Protective effects of mesenchymal stem cells with CXCR4 up-regulation in a rat renal transplantation model. PLoS One, 2013. 8(12): p. e82949.

37. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.

38. Yin, N., et al., Islet-1 promotes the cardiac-specific differentiation of mesenchymal stem cells through the regulation of histone acetylation. Int J Mol Med, 2014. 33(5): p. 1075-82.

39. Hajizadeh-Sikaroodi, S., et al., Lentiviral Mediating Genetic Engineered Mesenchymal Stem Cells for Releasing IL-27 as a Gene Therapy Approach for Autoimmune Diseases. Cell J, 2014. 16(3): p. 255-62.

40. He, H., et al., Mesenchymal Stem Cells Overexpressing Angiotensin-Converting Enzyme 2 Rescue Lipopolysaccharide-Induced Lung Injury. Cell Transplant, 2014.

41. Ma, H. C., et al., Targeted migration of mesenchymal stem cells modified with CXCR4 to acute failing liver improves liver regeneration. World J Gastroenterol, 2014. 20(40): p. 14884-94.

42. Yang, J. X., et al., CXCR4 receptor overexpression in mesenchymal stem cells facilitates treatment of acute lung injury in rats. J Biol Chem, 2015. 290(4): p. 1994-2006.

43. Zhu, Y., et al., Mesenchymal stem cell-based NK4 gene therapy in nude mice bearing gastric cancer xenografts. Drug Des Devel Ther, 2014. 8: p. 2449-62.

44. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.

45. Xu, W., et al., Basic fibroblast growth factor expression is implicated in mesenchymal stem cells response to light-induced retinal injury. Cell Mol Neurobiol, 2013. 33(8): p. 1171-9.

46. Li, W., et al., Bcl-2 engineered MSCs inhibited apoptosis and improved heart function. Stem Cells, 2007. 25(8): p. 2118-27.

47. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.

48. Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75.

49. Le Blanc, K., et al., Treatment of severe acute graft-versus-host disease with third party haploidentical mesenchymal stem cells. Lancet, 2004. 363(9419): p. 1439-41.

50. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.

51. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.

52. Reinisch, A., et al., Humanized system to propagate cord blood-derived multipotent mesenchymal stromal cells for clinical application. Regen Med, 2007. 2(4): p. 371-82.

53. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.

54. Lataillade, J. J., et al., New approach to radiation burn treatment by dosimetry-guided surgery combined with autologous mesenchymal stem cell therapy. Regen Med, 2007. 2(5): p. 785-94.

55. Seshareddy, K., D. Troyer, and M. L. Weiss, Method to isolate mesenchymal-like cells from Wharton's Jelly of umbilical cord. Methods Cell Biol, 2008. 86: p. 101-19.

56. Sensebe, L., Clinical grade production of mesenchymal stem cells. Biomed Mater Eng, 2008. 18(1 Suppl): p. S3-10.

57. Sotiropoulou, P. A., S. A. Perez, and M. Papamichail, Clinical grade expansion of human bone marrow mesenchymal stem cells. Methods Mol Biol, 2007. 407: p. 245-63.

58. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.

59. Zhang, X., et al., Cotransplantation of HLA-identical mesenchymal stem cells and hematopoietic stem cells in Chinese patients with hematologic diseases. Int J Lab Hematol, 2010. 32(2): p. 256-64.

60. Arrigoni, E., et al., Isolation, characterization and osteogenic differentiation of adipose-derived stem cells: from small to large animal models. Cell Tissue Res, 2009. 338(3): p. 401-11.

61. Grisendi, G., et al., GMP-manufactured density gradient media for optimized mesenchymal stromal/stem cell isolation and expansion. Cytotherapy, 2010. 12(4): p. 466-77.

62. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.

63. Sensebe, L., P. Bourin, and K. Tarte, Good manufacturing practices production of mesenchymal stem/stromal cells. Hum Gene Ther, 2011. 22(1): p. 19-26.

64. Capelli, C., et al., Minimally manipulated whole human umbilical cord is a rich source of clinical-grade human mesenchymal stromal cells expanded in human platelet lysate. Cytotherapy, 2011. 13(7): p. 786-801.

65. Ilic, N., et al., Manufacture of clinical grade human placenta-derived multipotent mesenchymal stromal cells. Methods Mol Biol, 2011. 698: p. 89-106.

66. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.

67. Timmins, N. E., et al., Closed system isolation and scalable expansion of human placental mesenchymal stem cells. Biotechnol Bioeng, 2012. 109(7): p. 1817-26.

68. Warnke, P. H., et al., A clinically-feasible protocol for using human platelet lysate and mesenchymal stem cells in regenerative therapies. J Craniomaxillofac Surg, 2013. 41(2): p. 153-61.

69. Fekete, N., et al., GMP-compliant isolation and large-scale expansion of bone marrow-derived MSC. PLoS One, 2012. 7(8): p. e43255.

70. Hanley, P. J., et al., Manufacturing mesenchymal stromal cells for phase I clinical trials. Cytotherapy, 2013. 15(4): p. 416-22.

71. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. 15(9): p. 1086-97.

72. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.

73. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.

74. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.

75. Martins, J. P., et al., Towards an advanced therapy medicinal product based on mesenchymal stromal cells isolated from the umbilical cord tissue: quality and safety data. Stem Cell Res Ther, 2014. 5(1): p. 9.

76. Iudicone, P., et al., Pathogen-free, plasma-poor platelet lysate and expansion of human mesenchymal stem cells. J Transl Med, 2014. 12: p. 28.

77. Skrahin, A., et al., Autologous mesenchymal stromal cell infusion as adjunct treatment in patients with multidrug and extensively drug-resistant tuberculosis: an open-label phase 1 safety trial. Lancet Respir Med, 2014. 2(2): p. 108-22.

78. Ikebe, C. and K. Suzuki, Mesenchymal stem cells for regenerative therapy: optimization of cell preparation protocols. Biomed Res Int, 2014. 2014: p. 951512.

79. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.

80. Swamynathan, P., et al., Are serum-free and xeno-free culture conditions ideal for large scale clinical grade expansion of Wharton's jelly derived mesenchymal stem cells? A comparative study. Stem Cell Res Ther, 2014. 5(4): p. 88.

81. Vaes, B., et al., Culturing protocols for human multipotent adult stem cells. Methods Mol Biol, 2015. 1235: p. 49-58.

82. Devito, L., et al., Wharton's jelly mesenchymal stromal/stem cells derived under chemically defined animal product-free low oxygen conditions are rich in MSCA-1(+) subpopulation. Regen Med, 2014. 9(6): p. 723-32.

1. A method of treating neurological manifestations of Zika Virus comprising the steps of: a) obtaining a patient suffering from Zika Virus neurological manifestation; b) administering a therapeutic dose of stem cells; c) assessing effect of said stem cell administration; and d) performing additional administrations of said stem cells based on neurological response achieved.

2. The method of claim 1, wherein said stem cells are selected from a group comprising of: a) adipose derived stem cells; b) embryonic stem cells; c) inducible pluripotent stem cells; d) hematopoietic stem cells; and e) mesenchymal stem cells.

3. The method of claim 2, wherein said mesenchymal stem cell is derived from tissue comprising a group selected from: a) Wharton's Jelly; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

4. The method of claim 3, wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

5. The method of claim 4, wherein said mesenchymal stem cells do not express substantial levels of HLA-DR, CD117, and CD45.

6. The method of claim 3, wherein said mesenchymal stem cells are generated from a pluripotent stem cell.

7. The method of claim 6, wherein said pluripotent stem cell is selected from a group comprising of: a) an embryonic stem cell; b) an inducible pluripotent stem cell; c) a parthenogenic stem cell; and d) a somatic cell nuclear transfer derived stem cell.

8. The method of claim 7, wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

9. The method of claim 7, wherein said inducible pluripotent stem cell possesses markers selected from a group comprising of: CD10, CD13, CD44, CD73, CD90, PDGFr-alpha, PD-L2, and HLA-A,B,C and possesses ability to undergo at least 40 doublings in culture, while maintaining a normal karyotype upon passaging.

10. The method of claim 7, wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

11. The method of claim 7, wherein said somatic cell nuclear transfer derived stem cells possess a phenotype negative for SSEA-1 and positive for SSEA-3, SSEA-4, TRA-1-60, TRA-1-81, and alkaline phosphatase.

12. The method of claim 6, wherein said mesenchymal stem cells are differentiated from a pluripotent stem cell source through culture in the presence of an inhibitor of the SMAD-2/3 pathway.

13. The method of claim 12, wherein said mesenchymal stem cells are differentiated from a pluripotent stem cell source through culture in the presence of an inhibitor nucleic acid targeting the SMAD-2/3 pathway.

14. The method of claim 13, wherein said nucleic acid inhibitor is selected from a group comprising of: a) an antisense oligonucleotide; b) a hairpin loop short interfering RNA; c) a chemically synthesized short interfering RNA molecule; and d) a hammerhead ribozyme.

15. The method of claim 13, wherein said inhibitor of the SMAD-2/3 pathway is a small molecule inhibitor.

16. The method of claim 15, wherein said small molecule inhibitor is SB-431542.

17. The method of claim 6, wherein a selection process is used to enrich for mesenchymal stem cells differentiated from said pluripotent stem cell population.

18. The method of claim 17, wherein said enrichment method comprises of positively selecting for cells expressing a marker associated with mesenchymal stem cells.

19. The method of claim 18, wherein said marker of mesenchymal stem cells is selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

20. The method of claim 1, wherein said immune modulatory cells are autologous, allogeneic or xenogeneic to the recipient.

21. The method of claim 2 wherein one or more cells are co-administered to said recipient based on specific need for immune modulation in said recipient.

22. The method of claim 2, wherein an antigen is administered in combination with immune modulatory cells.

23. The method of claim 1, wherein said stem cells is a cell obtained from a subepithelial layer of a mammalian umbilical cord tissue capable of self-renewal and culture expansion; wherein the isolated cell expresses at least three cell markers selected from the group consisting of CD29, CD73, CD90, CD166, SSEA4, CD9, CD44, CD146, or CD105; and wherein the isolated cell does not express at least three cell markers selected from the group consisting of CD45, CD34, CD14, CD79, CD106, CD86, CD80, CD19, CD117, Stro-1, or HLA-DR.

24. The method of claim 23, wherein said isolated cell expresses CD29, CD73, CD90, CD166, SSEA4, CD9, CD44, CD146, and CD105.

25. The method of claim 23, wherein said isolated cell does not express CD45, CD34, CD14, CD79, CD106, CD86, CD80, CD19, CD117, Stro-1, and HLA-DR.

26. The method of claim 23, wherein said isolated cell expresses one or more markers selected from a group comprising of: a) SOX2; b) OCT-4; c) NANOG; and d) KLF-4.

27. The method of claim 1, wherein said stem cells are treated with neurotrophic factors prior to administration.

28. The method of claim 27, wherein said neurotrophic factors are selected from a group comprising of: a) NGF; b) HGF-1; c) BDNF; and d) FGF-5.

29. The method of claim 1, wherein exosomes are derived from said stem cells and used together, or in substitution of said stem cells.

30. A method of treating neurological manifestations of Zika Virus through reducing microglial production of quinolinic acid through administration of mesenchymal stem cells.

31. The method of claim 1, wherein said mesenchymal stem cells are placentally derived and express CD34 upon isolation.
